(19) United States
(12) Patent Application Publication
(10) Pub. No.: US 2010/0120902 A1
(43) Pub. Date: May 13, 2010

(54) STANDARIZED BIOACTIVE HERBAL EXTRACTS

(75) Inventors: Chandra Kant Katiyar, Haryana (IN); Anil Kanaujia, Uttar Pradesh (IN); Rajeev Duggar, Rajasthan (IN); Satyapal Singh Yadav, Haryana (IN); Navin Sharma, Delhi (IN); Abhijit Ray, Delhi (IN); Rajkumar Shirumalla, Delhi (IN); Malini Bajpai, Uttar Pradesh (IN); Gyanesh Shukla, Uttar Pradesh (IN); Baddireddi Subhadra Lakshmi, Tamil Nadu (IN); Kontham Sanathkumar Vinaykumar, Tamil Nadu (IN)

Correspondence Address: Ranbaxy Inc., Intellectual Property Department, 600 College Road East, PRINCETON, NJ, 08540, US

(73) Assignee: RANBAXY LABORATORIES LIMITED, GURGAON, HARYANA (IN)

(21) Appl. No.: 12/532,815

(22) PCT Filed: Mar. 24, 2008
(86) PCT No.: PCT/IB08/51089

§ 371 Date: Jan. 8, 2010

(30) Foreign Application Priority Data
Mar. 23, 2007 (IN) 635/DEL/2007
Sep. 11, 2007 (IN) 1928/DEL/2007

(51) Int. Cl. A61K 31/352; A61P 29/00; A61P 19/02; A61P 19/00

(52) U.S. Cl. 514/453

(57) ABSTRACT

The present invention relates to standardized extracts of Boerhaavia diffusa, wherein the extracts have anti inflammatory and analgesic activities. The present invention also includes bioassay guided fractionation of Boerhaavia diffusa leading to the identification of bioactive markers; processes for the isolation of the bioactive markers; processes for the preparation of the extracts enriched with bioactive markers from Boerhaavia diffusa; pharmaceutical compositions comprising bioactive markers, or standardized extracts of Boerhaavia diffusa and methods of standardization of the extracts.

### FIELD OF THE INVENTION

The present invention relates to standardized extracts of Boerhaavia diffusa, wherein the extracts have anti inflammatory and analgesic activities. The present invention also includes bioassay guided fractionation of Boerhaavia diffusa leading to the identification of bioactive markers; processes for the isolation of the bioactive markers; processes for the preparation of the extracts enriched with bioactive markers, from Boerhaavia diffusa; pharmaceutical compositions comprising standardized extracts of Boerhaavia diffusa or bioactive markers; and methods of standardization of the extracts.

### BACKGROUND OF THE INVENTION

Inflammation is a pathological process characterized by injury or destruction of tissues, caused by a variety of cytologic and chemical reactions. It is usually manifested by typical signs of pain, heat, redness, swelling, and loss of function.

Inflammation plays a key role in many diseases such as arthritis, and there is increased evidence that atherosclerosis and Alzheimer disease also share uncontrolled inflammation as part of their etiology.

Inflammation has two major components, exudative and cellular. The exudative component involves the dilatation of upstream blood vessels and constriction of downstream blood vessels due to histamine, bradykinins or leukotrienes released from the injured tissue, thereby increasing the permeability of capillaries surrounding the injured tissue and exudation of fluid along with important proteins such as fibrin and immunoglobulins, thereby giving rise to edema or swelling.

The cellular component involves the migration of inflammatory cells (neutrophils, lymphocytes and macrophages) to injured/infected tissue for the immediate defense, protection or phagocytic action, which prevents further spreading the infection.

Macrophages stimulate the inflammatory responses of neutrophils, fibroblasts and endothelial cells in response to the infection by secreting various interleukins (IL) and tumor necrosis factor (TNF). Fibroblasts and endothelial cells respond to interleukin-1 (IL-1) and TNF by recruiting more immune cells to the site of inflammation. Further TNF-α (tumor necrosis factor-α), IL-1 and other cytokines activate endothelial cells to up regulate various adhesion molecule receptors viz. VCAM-1 (vascular cell adhesion molecule), ICAM-1 (intercellular adhesion molecule), E-selectin and L-selectin from various immune cells. Receptor activation further increases extravasations of nonspecific as well as specific immune cells.

The increased expression and release of TNF-α, IL-1β and nitric oxide (NO) cytokines further induce the expression and overproduction of various other inflammatory mediators such as cyclooxygenase 2 (cox-2), PGE2 (prostaglandin E2), ROS (reactive oxygen species), iNOS (inducible nitric oxide synthase) and IL-6 (interleukin-6). These mediators modulate important cellular functions including gene expression, DNA damage and cellular proliferation of immune and surrounding cells.

Tumor necrosis factor (TNF) and interleukin-1 (IL-1) are considered to be master cytokines in chronic, destructive arthritis. Analysis of cytokine patterns in early synovial biopsies of rheumatoid arthritis (RA) patients reveal a marked heterogeneity, with variable staining of TNF and IL-1β, suggesting their definitive role in pathogenesis of disease. Inhibition of IL-1β has shown the benefits in experimental arthritis and directed therapy for IL-1, with IL-1 receptor antagonist, mainly reduces erosions and is anti-inflammatory. Thus inhibition of inflammatory cytokines is empirical in management of inflammatory processes/diseases such as arthritis. An agent which has potential to block both the cytokines will be preferred therapy rather than agent targeting the single cytokine.

Inflammation is often associated with pain. Pain refers to the subjective, unpleasant sensation that accompanies damage or near-damage to tissues, though it can also occur in the absence of such damage, if the systems of nociception are not functioning properly. In simple terms, it is a physical and emotional symptom of being damaged or sick. The origin or source of pain may be cutaneous, somatic, visceral or others, such as neuropathic or phantom limb. The best treatment for most of the pain is to stop the damage that causes pain, however agents that are used to relieve the pain, i.e. non-steroidal anti-inflammatory drugs (NSAIDs), opoid analgesics and anti-depressant drugs act through different mechanisms.

Herbal medicines have emerged as a unique approach for meeting the need for safe, effective and relatively inexpensive new remedies for a variety of disorders. Herbal medicines represent the fastest growing segment among all of alternative medicine. These are produced in different forms, which range from crude, decocted herbs to refined, concentrated and standardized extracts. The health benefit from taking those herbals also varies with the quality of the products and the knowledge of consumers on the products. Some of the products have to be used under a physician's supervision, particularly those indicated for serious diseases although the majority of herbal medicines are generally safe.

Boerhaavia diffusa Linn (punarnava) (B. diffusa) is an ayurvedic medicinal plant used traditionally for the treatment of a number of diseases. Literature mentions, punarnava as bitter, astringent, diuretic, emetic, expectorant and cardiac stimulant. It is mentioned to be useful in anaemia, inflammations, heart diseases, asthma, opthalmia, leucorrhea etc (Warrier et al, Indian Medicinal Plants: A compendium of 500 species. Orient Longman Ltd, Madras, India 5: (1995), 132-134; Sharma et al, Database on Medicinal Plants used in Ayurveda, Central Council for Research in Ayurveda and Siddha medicine, Dept of ISM and H; Ministry of Health, Govt of India, New Delhi Vol I: (2001), 360-77).

Leaves of B. diffusa have been shown to possess antinociceptive and anti-inflammatory activities (Hiruma-Lima et al, Journal of Ethnopharmacology, 71: (2000), 267-274).

In-vitro studies have also suggested the immunomodulatory potential in root of B. diffusa. Ethanolic extract of root inhibited T cell mitogen phytohemagglutinin and concanavalin A-stimulated proliferation of human peripheral blood mononuclear cells (Mehrotra et al, Int Immunopharmacol., 2 (7): (2002), 987-996). Extract also inhibited human NK (natural killer) cell cytotoxicity, production of NO in mouse macrophage cells, IL-2 and TNF-α in human PBMCs (peripheral blood mononculear cells) (Mehrotra et al, Int Immunopharmacol. 2 (7): (2002), 987-96). However, Mungantiwar et al (J Ethnopharmacol., 65 (2): (1999), 125-31) observed in-vivo immunostimulatory activity of B. diffusa alkaloidal fraction without any in-vitro effect.

Further studies with chloroform and ethanol extracts of root gave anti-inflammatory activity to a pure compound Bd-I (eupalitin-3-O-beta-D-galactopyranoside) purified from the ethanolic extract (Pandey et al, Int Immunopharmacol., 5 (3): (2005), 541-53). Bd-I was reported to be equally or somewhere more effective than the parent ethanolic extract (Pandey et al, Int. Immunopharmacol., 5 (3): (2005), 541-53). Bd-I inhibited the production of IL-2 at the protein and mRNA transcript levels (phytohemagglutinin stimulated) and TNF-α production (lipo-polysaccharide induced) in human PBMCs. Bd-I was also shown to block the activation of DNA binding of nuclear factor-(kappa)B and transcription factor AP-1 (activating protein-1) (Pandey et al, Int Immunopharmacol., 5 (3): (2005), 541-53).

The compounds found in the root include Hentriacontane, β-Sitosterol, Ursolic acid (Misra and Tiwari, Phytochem., 10 (1971), 3318-19); virus inhibitor from the root extract (Verma et al, Can. J. Bot., 979 (57): 1214-17); Triacontanol, β-Sitosterol, β-ecdysone (Sufi et al, Planta Med., 44 (1982), 180); 5,7-dihydroxy-3′,4′-dimethoxy-6,8-dimethyl flavone (Gupta and Bahar, Ind. J. Chem., 23B (1984), 682-84). Hypoxanthine-9-L-arabinofuranoside (Purine nucleoside) (Ojewole and Adesina, Fitoterapia 56 (1): (1985), 31-36); Punarnavoside (Jain and Khanna, Ind. J. Chem., 28B (1989), 163-66); Boeravinone A, Boeravinone B, Boeravinone C, Boeravinone D, Boeravinone E, Boeravinone F (Rotenoids), Iiriodendrin (Lignans), Syringaresinol mono-β-D-glucoside (Kadota et al, Chem. Parm. Bull., 36 (2): (1988), 834-36; Kadota et al, Chem. Pharm. Bull., 36 (6): (1988), 2289-92; Kadota et al, Chem. Pharm. Bull., 37 (12): (1989), 3214-20; Kadota et al, Chem. Pharm. Bull., 38 (6): (1990), 1558-62; Kadota et al, Chem. Pharm. Bull., 39 (7): (1991), 1863-65); Borhavine (Dihydroisofurnanoxanthone) (Ahmed et al, Phytochemistry, 31 (12): (1992), 4382-84); Boerhavistreol (I), Boerhadiffusene (II), Diffusarotenoid, boerhavilanostenyl benzoate and Boeravinone A (Gupta et al, Ind. J. Chem., 37B (9): (1998), 912-17); Triacontenoic acid (Shrivastava and Shukla, Ind. Drugs 35 (2): (1998), 103-104); 5-methyleicos-4-ene, eicos-4-ene, 4-methyloctadec-3-ene and 4-methylnonadecylbenzene (Singh et al, J. Ind. Chem. Soc., 79 (11): (2002), 911-12).

One major problem in assuring the consistent quality of herbal supplements is caused by the natural variation of endogenous phytochemicals that occur in plants. The chemical “fingerprint” of a particular species of a plant can vary widely depending on the age of the plant, time of harvest, soil conditions, weather conditions, and other factors. It is reasonable to think that plants that have very different phytochemical profiles will have different therapeutic effects, even if the plants are from the same species.

Standardization of herbal extracts offers the batch-to-batch reproducibility of the final product. A standardized extract has a concentration of marker compound that is known to a high degree of accuracy, and because both the amount of plant material that is extracted and the amount of carrier that is added can be varied, it is possible to compensate for natural variability in the plant material. Also, if physicians have an informative knowledge about the amount of active components administered to the patients, the treatments following prognosis of the diseases can be monitored. Therefore, there is a need for standardized and reproducible extracts of Boerhaavia diffusa.

### SUMMARY OF THE INVENTION

In one aspect of the invention, there are provided standardized extracts of Boerhaavia diffusa.

In another aspect of the invention, a pharmaceutical composition comprising a standardized extract of Boerhaavia diffusa, Boeravinone B or Boeravinone E, along with one or more of pharmaceutically acceptable carriers, excipients or diluents is provided.

In another aspect of the invention, there is provided a process for the isolation of Boeravinone B and Boeravinone E from Boerhaavia diffusa.

In another aspect of the invention, a process for the preparation of extracts of Boerhaavia diffusa enriched with bioactive markers is provided.

In another aspect of the invention, there is provided a method for the standardization of extracts of Boerhaavia diffusa.

In another aspect of the invention, there is provided a method of treating inflammatory diseases, for example, rheumatoid arthritis, osteoarthritis, acute myoskeletal disorders, spondylosis, ankylosing spondylitis, bursitis, tendonitis, inflammatory lung disease, inflammatory bowel disease, atherosclerosis, systemic lupus erythematosus, multiple sclerosis, pelvic inflammatory disease or psoriasis, in a mammal comprising administering a therapeutically effective amount of Boeravinone B, Boeravinone E or a standardized extract of Boerhaavia diffusa.

In another aspect of the invention, there is provided a method of treating pain, for example, dental pain, muscular pain, neck pain, ear pain, joints pain, headache, abdominal pain, renal pain, pelvic pain, prolapsed intervertebral disc pain or neuropathic pain, in a mammal comprising administering a therapeutically effective amount of Boeravinone B, Boeravinone E or a standardized extract of Boerhaavia diffusa.

The details of one or more embodiments of the invention are set forth in the description below. Other features, objects and advantages of the inventions will be apparent from the description.

### BRIEF DESCRIPTION OF THE DRAWINGS

FIG. 1 shows flow diagram for the bioassay guided fractionation process.

FIG. 2 shows in-vitro Dose response curve for Mitogen induced Lymphocyte Proliferation Assay, wherein
7 denotes 0.1 μg/ml
6 denotes 1 μg/ml
5 denotes 10 μg/ml
4.6 denotes 20 μg/ml
4.3 denotes 50 μg/ml
4 denotes 100 μg/ml

FIG. 3 shows effect of Boerhavia diffusa methanol extract, and chloroform, butanol and aqueous fractions of the methanol extract on TNFα, IL-1β and iNOS mRNA expression in RAW 264.7 cells after 12 h of incubation, wherein, 355 bp, 388 by and 210 by are base pair marker bands for characteristic mRNA protein.

FIG. 4 shows effect of methanol extract and its chloroform fraction on in-vitro nitric oxide release from RAW 264.7 cells.

FIG. 5 shows effect of treatment on Phenyl-p-benzoquinone induced writhing in mice for analgesic efficacy, as described in Example 15(i) wherein ** signifies p<0.01.

FIG. 6 shows effect of treatment on carrageenan induced hyperalgesia in rats for analgesic efficacy, as described in Example 15(j) wherein * signifies p<0.05.

FIG. 7 shows effect of treatment on formalin induced Phase I and Phase II pain in mice for analgesic efficacy, as described in Example 15(k) wherein * signifies p<0.05, ** signifies p<0.01.

FIG. 8 shows effect of treatment on Complete Freund's adjuvant (CFA) induced hyperalgesia in rats for antihyperalgesic efficacy, as described in Example 15(l) wherein ** signifies p<0.01.

FIG. 9 shows effect of treatment on carrageenan induced paw edema in rats for anti-inflammatory efficacy, as described in Example 15(m) wherein * signifies p<0.05.

FIG. 10 shows effect of treatment on endotoxemia in female Balb/C mice for anti-inflammatory efficacy, as described in Example 15(n) wherein * signifies p<0.05.

FIG. 11 shows effect of treatment in air pouch model in rats for anti-inflammatory efficacy, as described in Example 15(o) wherein * signifies p<0.05, ** signifies p<0.01.

FIG. 12 shows effect of treatment on Complete Freund's adjuvant (CFA) induced arthritis in rats for antiarthritic efficacy, as described in Example 15(p) wherein * signifies p<0.05; ** signifies p<0.01.

FIG. 13 shows histopathological analysis of CFA induced arthritis in ankle joint of rats, as described in Example 15(q) wherein

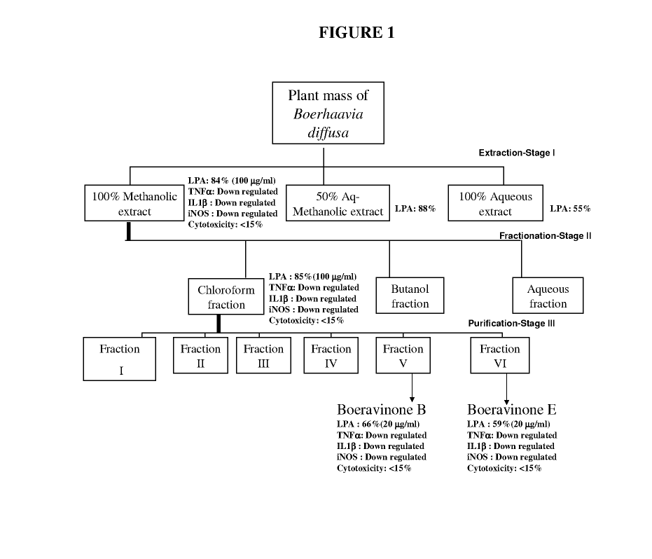

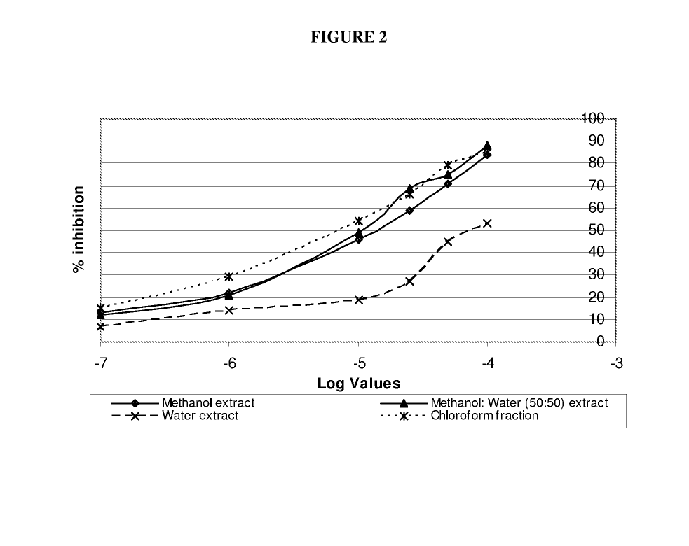

A) Shows complete Fruends Adjuvant (CFA) induced arthritis in ankle joint of Wistar rats wherein picture (A)a depicts the moderate inflammation of mixed population of neutrophils, macrophages and lymphocytes into the proliferated synovial membrane (dotted arrow), picture (A)b depicts that chloroform fraction 1200 mg/kg/day treated animals exhibited moderate reduction in inflammatory cell infiltration, picture (A)c shows that indomethacin, 0.2 mg/kg/day completely prevented the recruitment of inflammatory cells.

B) Shows in picture (B)a that moderate to marked fibro vascular proliferation of synovial membrane (Pannus) intruded into the joint space of ankle joint (solid arrow) in vehicle control group. But, the Pannus formation was mild in both Indomethacin and chloroform fraction 1200 mg/kg/day groups (pictures (B)b and (B)c).

C) Shows in picture (C)a that severe joint damage was noticed in vehicle treated animals with the resumption of bone and cartilage erosion as evidenced by more number of orthoclase cells ( [symbol] ). The inhibitory effect of chloroform fraction 1200 mg/kg/day and indomethacin on cartilage degradation was prominent as seen in pictures (C)b and (C)c, respectively. H & E refers to hematoxylin and eosin staining.

### DETAILED DESCRIPTION OF THE INVENTION

The invention provides a bioassay guided fractionation of plant mass of Boerhaavia diffusa leading to the identification and characterization of the bioactive markers. The process includes preparing different extracts of Boerhaavia diffusa, subjecting the extracts to the primary screening for bioactivity using lymphocyte proliferation inhibition assay, and further, evaluating the most active extract against secondary target assays (LPS stimulated TNF-α, IL-1β and NO, LTB₄ release from PBMC and mRNA expression for TNF-α, IL-1β and inducible nitric oxide synthase (iNOS) in RAW 264.7 cells). Under this approach, the active extract(s) are subjected to fractionation by one or more solvents, and each fraction is evaluated for the primary bioactivity assay. The active fraction is evaluated against secondary bioactivity assays and subjected to column chromatography for further fractionation and isolated fractions from active solvent fraction are evaluated for bioactivity using primary assay, the active fractions obtained are screened against secondary assays and the most active compounds isolated from these active fractions are characterized as Boeravinone B and Boeravinone E using spectroscopy.

The solvent for preparing different extracts and for fractionating the active extract may be alcohol, for example, methanol, ethanol, n-propanol, isopropanol or butanol; halogenated hydrocarbon; for example, chloroform, dichloromethane or dichloroethane; water; or mixture(s) thereof.

A new series of extracts enriched with bioactive markers is prepared, the enriched extracts are evaluated for the bioactivity using primary and secondary target assays and the most active extracts are evaluated for in-vivo anti-inflammatory and analgesic activity.

The invention provides processes for the isolation of Boeravinone B and Boeravinone E,

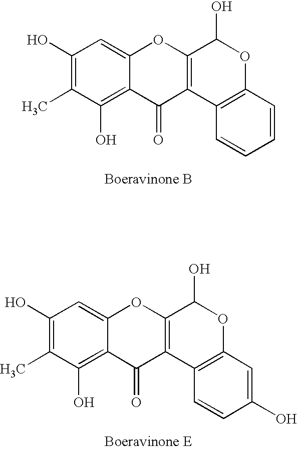

from Boerhaavia diffusa. The processes include, extracting the plant mass of Boerhaavia diffusa with one or more solvents, concentrating the extract, adding water to extract, partitioning the extract with one or more solvents and isolating Boeravinone B and Boeravinone E.

The powdered roots of Boerhaavia diffusa are extracted with one or more solvents selected from alcohol, for example, methanol, ethanol, n-propanol, isopropanol or butanol; ketone, for example, acetone or methyl isobutyl ketone; ester, for example, ethyl acetate or methyl acetate; halogenated hydrocarbon, for example, chloroform, dichloromethane or dichloroethane; nitrile, for example, acetonitrile; or mixture(s) thereof. The combined extracts are then concentrated under reduced pressure. The concentrated extracts are mixed with water, the residual solvent is skipped off and the aqueous layer is partitioned with one or more solvents selected from halogenated hydrocarbon, for example, dichloromethane, dichloroethane or chloroform; ester, for example, ethyl acetate or methyl acetate; alcohol, for example, butanol; ether, for example, diethyl ether; or mixture(s) thereof. The solvent layers are pooled together, concentrated under reduced pressure and the residue is subjected to column chromatographic purification. Elution is done with an increasing volume of methanol in chloroform. The fractions are collected separately and scanned for Boeravinone B and Boeravinone E presence by TLC using a mobile phase, for example, chloroform:methanol::90:10, 85:15 or 80:20. The fractions having Boeravinone B and Boeravinone E as observed by TLC pattern are combined and concentrated. The crude Boeravinone B and Boeravinone E are then crystallized from methanol from the respective fractions.

The invention also provides processes for the preparation of extracts of Boerhaavia diffusa enriched with bioactive markers. The processes include extracting the plant mass of Boerhaavia diffusa with one or more solvents from non polar to polar range, and drying the extract, or extracting the plant mass of Boerhaavia diffusa with one or more solvents from non polar to polar range, adding water and partitioning the extract with one or more solvents from non polar to polar range, and drying the extract.

The solvents for extraction may be alcohol, for example, methanol, ethanol, n-propanol, isopropanol or butanol; ketone, for example, acetone or methyl isobutyl ketone; ester, for example, methyl acetate or ethyl acetate; halogenated hydrocarbon, for example, chloroform, dichloromethane or dichloroethane; water; or mixture(s) thereof.

The solvents for partitioning may be halogenated hydrocarbon, for example, chloroform, dichloromethane or dichloroethane; ester, for example, ethyl acetate or methyl acetate; alcohol, for example, butanol; ether, for example, diethyl ether; or mixture(s) thereof.

Pulverized Boerhaavia diffusa roots are charged into the extractor followed by addition of one or more solvents such as alcohols, for example, methanol, ethanol, n-propanol, isopropanol or butanol; ketones, for example, acetone or methyl isobutyl ketones; esters, for example, methyl acetate or ethyl acetate; halogenated hydrocarbons, for example, chloroform, dichloromethane or dichloroethane; water; or mixture(s) thereof. The mixture is heated and the extracts are combined, concentrated and dried in vacuum oven.

Alternatively, pulverized Boerhaavia diffusa roots are charged into the extractor and one or more solvents such as alcohols, for example, methanol, ethanol, n-propanol, isopropanol or butanol; ketones, for example, acetone or methyl isobutyl ketones; esters, for example, ethyl acetate or methyl acetate; or mixture(s) thereof, are added. The mixture is heated and the extracts are combined and concentrated. Water is added to the extract(s) and one or more solvents such as halogenated hydrocarbon, for example, chloroform, dichloromethane, dichloroethane or mixture(s) thereof, are added to obtain organic and aqueous fractions. The organic fractions are combined, concentrated and dried in vacuum oven. The aqueous fraction is mixed with one or more solvents, for example, an alcohol such as butanol; to obtain organic and aqueous fractions. The organic and aqueous fractions are concentrated and dried in vacuum oven.

Alternatively, pulverized Boerhaavia diffusa roots are charged into the extractor and one or more solvents such as alcohol, for example, methanol, ethanol, n-propanol, isopropanol or butanol; ketone, for example, acetone or methyl isobutyl ketone; ester, for example, ethyl acetate or methyl acetate; or mixture(s) thereof, are added. The mixture is kept at room temperature for about 20 hours. The extracts are combined and concentrated. Water is added to the extract(s) and one or more solvents such as halogenated hydrocarbon, for example, chloroform, dichloromethane, dichloroethane or mixture(s) thereof, are added to obtain organic and aqueous fractions. The organic fractions are combined, concentrated and dried in vacuum oven. The aqueous fraction is mixed with one or more solvents such as alcohol, for example, butanol; to obtain organic and aqueous fractions. The organic and aqueous fractions are concentrated and dried in vacuum oven.

The invention provides standardized extracts of Boerhaavia diffusa and methods for the standardization of extracts, wherein the methods include detection and quantification of bioactive markers, for example, Boeravinone B and/or Boeravinone E.

HPLC method for the detection and quantification of bioactive markers, includes diluting the extract(s) in one or more solvents, sonicating the solution, filtering the supernatant liquid to form a test solution, injecting the test solution in a chromatographic column, running test chromatogram using a mobile phase, scanning, detecting the bioactive markers in the extract(s) by matching retention times of these bioactive markers in the test chromatogram with that of standard chromatogram and quantifying the bioactive markers.

The extract(s) may be diluted in a solvent such as alcohol, for example, methanol, ethanol, n-propanol or isopropanol; nitrile, for example, acetonitrile; or mixture(s) thereof.

The test chromatogram may be run in a mobile phase comprising one or more solvents such as alcohol, for example, methanol or ethanol; nitrile, for example, acetonitrile; water; or mixture(s) thereof and optionally one or more buffers, for example, formic acid, trifluoro acetic acid, ortho-phosphoric acid, ammonium acetate, sodium perchlorate, potasium dihydrogen orthophosphate, dipotasium hydrogen orthophosphate, sodium dihydrogen orthophosphate, disodium hydrogen orthophosphate, diammonium hydrogen orthophosphate, ammonium dihydrogen orthophosphate, ammonium formate, tetramethyl ammonium hydroxide, tetrabutyl ammonium hydroxide, tetrabutyl ammonium hydrogen sulphate or mixture(s) thereof.

Each of the standard chromatograms may be obtained by preparing standard bioactive marker solutions by dissolving bioactive markers separately in one or more solvents, injecting the standard bioactive marker solutions separately in chromatographic column, running standard chromatogram using a mobile phase and scanning.

The preparation of standard bioactive marker solutions may be carried out by dissolving the bioactive markers separately in one or more solvents such as alcohol, for example, methanol, ethanol, n-propanol or isopropanol; nitrile, for example, acetonitrile; or mixture(s) thereof. The solution may be sonicated and then made up to a desired fixed volume using the same solvent.

The standard chromatogram may be run in a mobile phase comprising one or more solvents such as alcohol, for example, methanol or ethanol; nitrile, for example, acetonitrile; water; or mixture(s) thereof and optionally one or more buffers, for example, formic acid, trifluoro acetic acid, ortho-phosphoric acid, ammonium acetate, sodium perchlorate, potasium dihydrogen orthophosphate, dipotasium hydrogen orthophosphate, sodium dihydrogen orthophosphate, disodium hydrogen orthophosphate, diammonium hydrogen orthophosphate, ammonium dihydrogen orthophosphate, ammonium formate, tetramethyl ammonium hydroxide, tetrabutyl ammonium hydroxide, tetrabutyl ammonium hydrogen sulphate or mixture(s) thereof.

The scanning may be done at the wavelength of from about 273 nm to 277 nm.

HPLC system used is a gradient system attached with PDA detector. Column used is C₁₈, 150×4.6 mm 5μ. (Purospher^{R} Star) or equivalent. Run time is from about 0 minutes to 65 minutes.

The percentage content of the bioactive markers in the test sample may be calculated as follows:

$$= \frac{{A}_{SPL}}{{A}_{STD}} \times \frac{{D}_{SPL}}{{D}_{STD}} \times \frac{{W}_{STD}}{{W}_{SPL}} \times \frac{P}{100} \times 100$$

wherein,

A_{SPL}—Average peak area corresponding to bioactive markers from the sample chromatograph

A_{STD}—Average peak Area corresponding to bioactive markers from the standard chromatograph

D_{SPL}—Dilution of test solution

D_{STD}—Dilution of reference standard solution

W_{STD}—wt. of reference standard taken in mg

W_{SPL}—wt. of test sample taken in mg

P—Purity of the reference standard

Boeravinone B and Boeravinone E are isolated from plant mass of Boerhaavia diffusa and each batch of the extract is standardized to contain 0.1%-4.0% of Boeravinone B and 0.05%-3.0% of Boeravinone E, respectively.

The extracts of Boerhaavia diffusa include (a) the extracts obtained by extraction of plant mass of Boerhaavia diffusa with one or more solvents, and (b) the fractions obtained by partitioning of the extracts of step (a) with one or more solvents.

The standardized extract may be prepared by extracting the plant mass of Boerhaavia diffusa with one or more solvents from non polar to polar range, and drying the extract, or extracting the plant mass of Boerhaavia diffusa with one or more solvents from non polar to polar range, adding water and partitioning the extract with one or more solvents from non polar to polar range, and drying the extract.

The solvents for extraction may be alcohol, for example, methanol, ethanol, n-propanol, isopropanol or butanol; ketone, for example, acetone or methyl isobutyl ketone; ester, for example, methyl acetate or ethyl acetate; halogenated hydrocarbon, for example, chloroform, dichloromethane or dichloroethane; water; or mixture(s) thereof.

The solvents for partitioning may be halogenated hydrocarbon, for example, chloroform, dichloromethane or dichloroethane; ester, for example, ethyl acetate or methyl acetate; alcohol, for example, butanol; ether, for example, diethyl ether; or mixture(s) thereof.

In-vitro as well as in-vivo anti-inflammatory and analgesic activities of the extracts of Boerhaavia diffusa; bioactive markers, Boeravinone B and Boeravinone E; and their mechanism are also provided.

The extracts of Boerhaavia diffusa and bioactive markers, Boeravinone B and Boeravinone E, may potentially treat inflammatory diseases, for example, rheumatoid arthritis, osteoarthritis, acute myoskeletal disorders, spondylosis, ankylosing spondylitis, bursitis, tendonitis, inflammatory lung disease, inflammatory bowel disease, atherosclerosis, systemic lupus erythematosus, multiple sclerosis, pelvic inflammatory disease or psoriasis.

The extracts of Boerhaavia diffusa and bioactive markers, Boeravinone B and Boeravinone E, may also treat pain of various origins, for example, dental pain, muscular pain, neck pain, ear pain, joints pain, headache, abdominal pain, renal pain, pelvic pain, prolapsed intervertebral disc pain or neuropathic pain or pain associated with other diseases.

Pharmaceutical compositions comprising standardized extracts of Boerhaavia diffusa, Boeravinone B or Boeravinone E, along with one or more of pharmaceutically acceptable carriers, excipients or diluents are provided, which may be administered to a mammal for treatment of inflammatory diseases or pain by any route, which effectively transports the active compound to the appropriate or desired site of action such as oral, nasal, pulmonary, transdermal or parenteral (rectal, subcutaneous, intravenous, intraurethral, intramuscular or intranasal). The choice of pharmaceutical carrier, excipient or diluent can be made with regard to the intended route of administration and standard pharmaceutical practice.

The term “bioactive markers” refers to biologically active chemical compounds which are present in the plant mass of Boerhaavia diffusa or its extract and have been used for standardization of the extract.

“Plant mass of Boerhaavia diffusa” refers to roots of the plant, aerial parts of the plant or whole plant.

“A standardized extract of Boerhaavia diffusa” refers to an extract of Boerhaavia diffusa, wherein bioactive markers are detected and quantified. The extracts of the present invention are obtained by extraction or partitioning with the solvents and the solvents are removed to a level acceptable in accordance with FDA ICH guidelines.

While the following examples are provided to certain embodiments of the invention, they are not intended to be limiting to the scope of the invention.

### Examples

### Example 1

### Preparation of Methanol Extract from Boerhaavia diffusa

Pulverized Boerhaavia diffusa roots (100 kg) were charged into the extractor. Methanol (300 liter) was added into the extractor and heating was done at 50° C. for about 4 hours. The extract was filtered and stored in a container. Again, 200 liter of methanol was added into the extractor and heating was done at 50° C. for about 4 hours. The extract was filtered and stored in a container. Again, 200 liter of methanol was added into the extractor and heating was done at 50° C. for about 4 hours. The methanolic extracts were combined and concentrated to maximum under reduced pressure at low temperature. The extract was down loaded into stainless steel (SS) trays and dried in vacuum oven at room temperature for about 16-18 hours.

Yield=6.27%

### Example 2a

### Preparation of Chloroform Fraction from Boerhaavia diffusa

Pulverized Boerhaavia diffusa roots (100 kg) were charged into the extractor. Methanol (300 liter) was added into the extractor and heating was done at 50° C. for about 4 hours. The extract was filtered and stored in a container. Again 200 liter of methanol was added into the extractor and heating was done at 50° C. for about 4 hours. The extract was filtered and stored in a container. Again, 200 liter of methanol was added into the extractor and heating was done at 50° C. for about 4 hours. Methanolic extracts were combined and concentrated to maximum under reduced pressure at low temperature. Water (300 liter) was added into the extractor containing methanolic extract and stirring was done at room temperature for about half an hour. Chloroform (100 liter) was added and stirring was done for about a half minute. The mixture was allowed to settle for about half an hour and the chloroform layer was separated into a container. This process was repeated for four more times and all the chloroform layers were collected in the container and passed over the sodium sulphate bed to dry it. The chloroform fraction was concentrated at 40° C. under reduced pressure, down loaded into SS trays and dried in vacuum oven at room temperature for about 16-18 hours.

Yield=0.38%

### Example 2b

### Preparation of Chloroform Fraction from Boerhaavia diffusa

Pulverized Boerhaavia diffusa roots (100 kg) were charged into the extractor. Methanol (300 liter) was added into the extractor and the mixture was kept at room temperature for about 20 hours. The extract was filtered and stored in a container. Again, methanol (200 liter) was added into the extractor and the mixture was kept at room temperature for about 20 hours. The extract was filtered and stored in a container. Methanol (200 liter) was again added into the extractor and the mixture was kept at room temperature for about 20 hours. The extract was filtered and stored in a container. Again, 200 liter of methanol was added into the extractor and the mixture was kept at room temperature for about 20 hours. The extract was filtered and stored in a container. All the methanolic extracts were combined and concentrated to maximum under reduced pressure at low temperature. Water (50 liter) was added into the extractor containing concentrated methanolic extract and the mixture was stirred at room temperature for about half an hour. Chloroform (50 liter) was added and the mixture was stirred for about a half minute. The mixture was allowed to settle for about half an hour and the chloroform layer was separated into a container. The process was repeated for four more times and all the chloroform layers were collected in a container. The chloroform fraction was passed over sodium sulphate bed to dry it and concentrated at 40° C. under reduced pressure. The fraction was downloaded into SS trays and dried in vacuum oven at room temperature for about 16-18 hours.

Yield=0.35%-0.45%

### Example 2c

### Preparation of Butanol Fraction from Boerhaavia diffusa

Pulverized Boerhaavia diffusa roots (100 kg) were charged into the extractor. Methanol (300 liter) was added into the extractor and the mixture was kept at room temperature for about 20 hours. The extract was filtered and stored in a container. Again, methanol (200 liter) was added into the extractor and the mixture was kept at room temperature for about 20 hours. The extract was filtered and stored in a container. Methanol (200 liter) was again added into the extractor and the mixture was kept at room temperature for about 20 hours. The extract was filtered and stored in a container. Again, 200 liter of methanol was added into the extractor and the mixture was kept at room temperature for about 20 hours. The extract was filtered and stored in a container. All the methanolic extracts were combined and concentrated to maximum under reduced pressure at low temperature. Water (50 liter) was added into the extractor containing concentrated methanolic extract and the mixture was stirred at room temperature for about half an hour. Chloroform (50 liter) was added and the mixture was stirred for about a half minute. The mixture was allowed to settle for about half an hour and the chloroform layer was separated into a container. The process was repeated for four more times. Butanol (50 liter) was added to aqueous layer and the mixture was stirred for about half minute. The mixture was allowed to settle for about half an hour and butanol layer was separated into a container. The process was repeated for two more times and all the butanol layers were collected in a container. The butanol fraction was dried over sodium sulphate bed and concentrated at 40° C. under reduced pressure. It was downloaded into SS trays and dried in vacuum oven at room temperature for about 16-18 hours.

Yield=1.2%-1.5%

### Example 2d

### Preparation of Aqueous Fraction from Boerhaavia diffusa

Pulverized Boerhaavia diffusa roots (100 kg) were charged into the extractor. Methanol (300 liter) was added into the extractor and the mixture was kept at room temperature for about 20 hours. The extract was filtered and stored in a container. Again, methanol (200 liter) was added into the extractor and the mixture was kept at room temperature for about 20 hours. The extract was filtered and stored in a container. Methanol (200 liter) was again added into the extractor and the mixture was kept at room temperature for about 20 hours. The extract was filtered and stored in a container. Again, 200 liter of methanol was added into the extractor and the mixture was kept at room temperature for about 20 hours. The extract was filtered and stored in a container. All the methanolic extracts were combined and concentrated to maximum under reduced pressure at low temperature. Water (50 liter) was added into the extractor containing concentrated methanolic extract and the mixture was stirred at room temperature for about half an hour. Chloroform (50 liter) was added and the mixture was stirred for about half minute. The mixture was allowed to settle for about half an hour and the chloroform layer was separated into a container. The process was repeated for four more times. Butanol (50 liter) was added to aqueous layer and the mixture was stirred for about half minute. The mixture was allowed to settle for about half an hour and the butanol layer was separated into a container. The process was repeated for two more times. The aqueous layer was concentrated at 40° C. under reduced pressure and downloaded into SS trays and dried in vacuum oven at room temperature for about 16-18 hours.

Yield=3.5%-4.0%

### Example 3

### Preparation of Acetone Extract from Boerhaavia diffusa

Pulverized Boerhaavia diffusa roots (100 kg) were charged into the extractor. Acetone (300 liter) was added into the extractor and heating was done at 45° C. for about 4 hours. The extract was filtered and stored in a container. Again, 200 liter of acetone was added into the extractor and heating was done at 45° C. for about 4 hours. The extract was filtered and stored in a container. Again, 200 liter of acetone was added into the extractor and heating was done at 45° C. for about 4 hours. Acetone extracts were combined and concentrated to maximum under reduced pressure at low temperature, down loaded the extract into SS trays and dried in vacuum oven at room temperature for about 16-18 hours.

Yield=0.6%

### Example 4

### Preparation of (Methanol:Ethyl acetate::50:50) Extract from Boerhaavia diffusa

Pulverized Boerhaavia diffusa roots (100 kg) were charged into the extractor. A mixture of methanol:ethyl acetate (150 liter: 150 liter) was added into the extractor and heating was done at 50° C. for about 4 hours. The extract was filtered and stored in a container. Again, a mixture of methanol:ethyl acetate (100 liter: 100 liter) was added into the extractor and heating was done at 50° C. for about 4 hours. The extract was filtered and stored in a container. Again, a mixture of methanol:ethyl acetate (100 liter: 100 liter) was added into the extractor and heating was done at 50° C. for about 4 hours. Methanol: ethyl acetate extracts were combined and concentrated to maximum under reduced pressure at low temperature, down loaded the extract into SS trays and dried in vacuum oven at room temperature for about 16-18 hours.

Yield=2.82%

### Example 5

### Preparation of Aqueous Extract from Boerhaavia diffusa

Pulverized Boerhaavia diffusa roots (100 kg) were charged into the extractor. Water (300 liter) was added into the extractor and heating was done at 50° C. for about 4 hours. The extract was filtered and stored in a container. Again, water (200 liter) was added into the extractor and heating was done at 50° C. for about 4 hours. The extract was filtered and stored in a container. Again, water (200 liter) was added into the extractor and heating was done at 50° C. for about 4 hours. The aqueous extracts were combined and concentrated to maximum under reduced pressure at low temperature, down loaded the extract into SS trays and dried in vacuum oven at room temperature for about 16-18 hours.

Yield=3.10%

### Example 6

### Preparation of (Methanol:Water::50:50) Extract from Boerhaavia diffusa

Pulverized Boerhaavia diffusa roots (100 kg) were charged into the extractor. A mixture of methanol:water (150 liter: 150 liter) was added into the extractor and heating was done at 50° C. for about 4 hours. The extract was filtered and stored in a container. Again, a mixture of methanol:water (100 liter: 100 liter) was added into the extractor and heating was done at 50° C. for about 4 hours. The extract was filtered and stored in a container. Again, a mixture of methanol:water (100 liter: 100 liter) was added into the extractor and heating was done at 50° C. for about 4 hours. The hydro alcoholic extracts were combined and concentrated to maximum under reduced pressure at low temperature, down loaded the extract into SS trays and dried in vacuum oven at room temperature for about 16-18 hours.

Yield=5.65%

### Example 7

### Preparation of Chloroform Extract from Boerhaavia diffusa

Pulverized Boerhaavia diffusa roots (100 kg) were charged into the extractor. Chloroform (300 liter) was added into the extractor and heating was done at 45° C. for about 4 hours. The extract was filtered and stored in a container. Again, chloroform (200 liter) was added into the extractor and heating was done at 45° C. for about 4 hours. The extract was filtered and stored in a container. Again, chloroform (200 liter) was added into the extractor and heating was done at 45° C. for about 4 hours. Chloroform extracts were combined and concentrated to maximum under reduced pressure at low temperature, down loaded the extract into SS trays and dried in vacuum oven at room temperature for about 16-18 hours.

Yield=0.57%

### Example 8

### Preparation of (Chloroform:Methanol::50:50) Extract from Boerhaavia diffusa

Pulverized Boerhaavia diffusa roots (100 kg) were charged into the extractor. A mixture of chloroform:methanol (150 liter: 150 liter) was added into the extractor and heating was done at 50° C. for about 4 hours. The extract was filtered and stored in a container. Again, a mixture of chloroform:methanol (100 liter: 100 liter) was added into the extractor and heating was done at 50° C. for about 4 hours. The extract was filtered and stored in a container. Again, a mixture of chloroform:methanol (100 liter: 100 liter) was added into the extractor and heating was done at 50° C. for about 4 hours. Chloroform:methanol extracts were combined and concentrated to maximum under reduced pressure at low temperature, down loaded the extract into SS trays and dried in vacuum oven at room temperature for about 16-18 hours.

Yield=3.47%

### Example 9

### Preparation of (Methanol:Acetone::50:50) Extract from Boerhaavia diffusa

Pulverized Boerhaavia diffusa roots (100 kg) were charged into the extractor. A mixture of methanol:acetone (150 liter: 150 liter) was added into the extractor and heating was done at 50° C. for about 4 hours. The extract was filtered and stored in a container. Again, a mixture of methanol:acetone (100 liter: 100 liter) was added into the extractor and heating was done at 50° C. for about 4 hours. The extract was filtered and stored in a container. Again, a mixture of methanol:acetone (100 liter: 100 liter) was added into the extractor and heating was done at 50° C. for about 4 hours. Methanol:acetone extracts were combined and concentrated to maximum under reduced pressure at low temperature, down loaded the extract into SS trays and dried in vacuum oven at room temperature for about 16-18 hours.

Yield=3.50%

### Example 10

### Preparation of (Methanol:Chloroform::10:90) Extract from Boerhaavia diffusa

Pulverized Boerhaavia diffusa roots (100 kg) were charged into the extractor. A mixture of methanol:chloroform (30 liter: 270 liter) was added into the extractor and heating was done at 50° C. for about 4 hours. The extract was filtered and stored in a container. Again, a mixture of methanol:chloroform (20 liter: 180 liter) was added into the extractor and heating was done at 50° C. for about 4 hours. The extract was filtered and stored in a container. Again, a mixture of methanol:chloroform (20 liter: 180 liter) was added into the extractor and heating was done at 50° C. for about 4 hours. Methanol: chloroform extracts were combined and concentrated to maximum under reduced pressure at low temperature, down loaded the extract into SS trays and dried in vacuum oven at room temperature for about 16-18 hours.

Yield=0.95%

### Example 11

### Preparation of (Methanol:Chloroform::20:80) Extract from Boerhaavia diffusa

Pulverized Boerhaavia diffusa roots (100 kg) were charged into the extractor. A mixture of methanol:chloroform (60 liter: 240 liter) was added into the extractor and heating was done at 50° C. for about 4 hours. The extract was filtered and stored in a container. Again, a mixture of methanol:chloroform (40 liter: 160 liter) was added into the extractor and heating was done at 50° C. for about 4 hours. The extract was filtered and stored in a container. Again, a mixture of methanol:chloroform (40 liter: 160 liter) was added into the extractor and heating was done at 50° C. for about 4 hours. Methanol:chloroform extracts were combined and concentrated to maximum under reduced pressure at low temperature, down loaded the extract into SS trays and dried in vacuum oven at room temperature for about 16-18 hours.

Yield=1.81%

### Example 12

### Isolation of Boeravinone B from Boerhaavia diffusa

Powdered Boerhaavia diffusa roots (1.0 Kg) were macerated with methanol (5.0 liters) at room temperature for about 24 hours and filtered. The extract was collected in a container. Again, methanol (2.0 liters) was added in the marc and maceration was done at room temperature for about 24 hours and filtration was done. The extract was collected in a container. Again, methanol (2.0 liters) was added in the marc and maceration was done at room temperature for about 24 hours and filtration was done. The extract was collected in a container. All the extracts were combined and concentrated under reduced pressure to ¼^{th} of its original volume. Equal volume of water was added and the residual methanol was skipped off on rotary evaporator. The aqueous layer was partitioned with chloroform for three times. Chloroform layers were combined and passed through the bed of sodium sulphate to dry it and concentration was done under reduced pressure. The material obtained was chromatographed over silica gel (100-200 mesh) and eluted with increasing volume of methanol in chloroform and different fractions were collected. Each fraction was observed by thin layer chromatography using mobile phase [chloroform:methanol (90:10, 85:15, 80:20)] and detected by visualizing under UV-254 nm. All the fractions having Boeravinone B as observed by their TLC pattern were combined, concentrated under reduced pressure and Boeravinone B was crystallized from methanol.

Yield: 0.004%

### Example 13

### Isolation of Boeravinone E from Boerhaavia diffusa

Powdered Boerhaavia diffusa roots (1.0 Kg) were macerated with methanol (5.0 liters) at room temperature for about 24 hours and filtered. The extract was collected in a container. Methanol (2.0 liters) was added in the marc and maceration was done at room temperature for about 24 hours and filtration was done. The extract was collected in a container. Methanol (2.0 liters) was added again in the marc and maceration was done at room temperature for about 24 hours and filtration was done. The extract was collected in a container. All the extracts were combined and concentration was done under reduced pressure to ¼^{th} of its original volume. Equal volume of water was added and the residual methanol was skipped off on rotary evaporator. The aqueous layer was partitioned for three times with chloroform. The chloroform layers were combined and passed through the bed of sodium sulphate to dry and concentrated under reduced pressure. The material obtained was chromatographed over silica gel (100-200 mesh) and eluted with increasing volume of methanol in chloroform and different fractions were collected. Each fraction was observed by thin layer chromatography using mobile phase [chloroform:methanol (90:10, 85:15, 80:20)] and detected by visualizing under UV -254 nm. All the fractions having Boeravinone E as observed by their TLC pattern were combined, concentrated under reduced pressure and Boeravinone E was crystallized from methanol.

Yield: 0.002%

### Example 14

### HPLC Method for the Detection and Quantification of Boeravinone B and Boeravinone E

a. Preparation of Reference Standard Solutions

(i) Boeravinone B

Boeravinone B reference standard (1.0 mg) was weighed in a 10 ml volumetric flask. Methanol (5.0 ml) was added, sonication was done in an ultrasonic water bath to dissolve and the volume was made up with methanol. The resulting solution was used as reference standard solution for Boeravinone B.

(ii) Boeravinone E

Boeravinone E reference standard (6.3 mg) was weighed in a 10 ml volumetric flask. Methanol (5.0 ml) was added, sonication was done in an ultrasonic water bath to dissolve and the volume was made up with methanol. The resulting solution was used as reference standard solution for Boeravinone E.

b. Preparation of Test Solutions

Boeravinone B and Boeravinone E

Extracts (40.0 mg) (examples 1 to 11) were weighed separately, in volumetric flasks (10 mL). Methanol (5 mL) was added to the extracts and sonication was done in an ultrasonic water bath for about 15 minutes. Cooling was done at room temperature and the volume was made up with methanol. Filtration was done through 0.45μ membrane filter and the resulting solutions were used as test solutions.

c. Detection and Quantification

Boeravinone B

Standard solutions (20 μL) and test solutions (40 μL, in case of methanol, methanol:chloroform::50:50, methanol:acetone::50:50, methanol:ethyl acetate::50:50, aqueous, methanol:water::50:50 and chloroform extracts) and test solutions (20 μL, in case of acetone, chloroform fraction, methanol:chloroform::10:90 and methanol:chloroform::20:80 extracts) were injected twice separately and the chromatograms were obtained.

Typical HPLC Conditions

Instrument: A Gradient High Performance Liquid Chromatographic System attached with PDA detector (Waters with class EMPOWER software)

Mobile Phase: Water:Methanol

Column: C_{18,} 150 mm×4 6 mm, 5μ (Purospher^{R} Star) or equivalent

Column Temp: 30° C.

Detector: PDA Detector

Wavelength For Recording The Chromatogram: 273 nm

Flow Rate: 10 mL/minutes

Injection Volume: 20 μL

Run Time: 65 minutes

Retention Time: 32.3 minutes

| Time | Flow Rate (ml/min) | Water | Methanol |
|---|---|---|---|
| 0 | 1 | 70 | 30 |
| 40 | 1 | 10 | 90 |
| 50 | 1 | 10 | 90 |
| 55 | 1 | 70 | 30 |
| 65 | 1 | 70 | 30 |

### Boeravinone E

Standard solutions (5 μL) and test solutions (40 μL, in case of methanol, methanol:chloroform::50:50, methanol:chloroform::10:90, methanol:acetone::50:50, methanol:ethyl acetate::50:50, aqueous, methanol:water::50:50, chloroform fraction, chloroform and acetone extracts) and test solutions (20 μL, in case of methanol:chloroform::20:80 extract) were injected twice separately and the chromatograms were obtained.

### Typical HPLC Conditions

Instrument: A Gradient High Performance Liquid Chromatographic System attached with PDA detector (Waters with class EMPOWER software)

Mobile Phase: Water:Methanol

Column: C_{18,} 150 mm×4 6 mm, 5μ (Purospher^{R} Star) or equivalent

Column Temp: 30° C.

Detector: PDA Detector

Wavelength For Recording The Chromatogram: 277 nm

Flow Rate: 1.0 mL/min

Injection Volume: 20 μL

Run Time: 65 minutes

Retention Time: 25 7 minutes

| Time | Flow Rate (ml/min) | Water | Methanol |
|---|---|---|---|
| 0 | 1 | 70 | 30 |
| 40 | 1 | 10 | 90 |
| 50 | 1 | 10 | 90 |
| 55 | 1 | 70 | 30 |
| 65 | 1 | 70 | 30 |

### Calculations

The percentage content of Boeravinone B and Boeravinone E was calculated as follows

$$= \frac{{A}_{SPL}}{{A}_{STD}} \times \frac{{D}_{SPL}}{{D}_{STD}} \times \frac{{W}_{STD}}{{W}_{SPL}} \times \frac{P}{100} \times 100$$

### Where,

A_{SPL}—Average peak area corresponding to Boeravinone B and Boeravinone E from the sample chromatograph

A_{STD}—Average peak Area corresponding to Boeravinone B and Boeravinone E from the standard chromatograph

D_{SPL}—Dilution of test solution

D_{STD}—Dilution of reference standard solution

W_{STD}—wt. of reference standard taken in mg

W_{SPL}—wt. of test sample taken in mg

P—Purity of the reference standard

Percentage Content of Boeravinone B and Boeravinone E in Different Extracts

| | Boerhaavia diffusa | % content of | % content of |
|---|---|---|---|
| Example | extract | Boeravinone B | Boeravinone E |
| 1 | Methanol extract | not less than 0.29 | not less than 0.18 |
| 2a and 2b | Chloroform fraction | not less than 1.17 | not less than 0.94 |
| 3 | Acetone extract | not less than 1.14 | not less than 0.83 |
| 4 | (Methanol:ethyl acetate::50:50) extract | not less than 0.66 | not less than 0.58 |
| 7 | Chloroform extract | not less than 0.57 | Nil |
| 8 | (Chloroform:methanol::50:50) extract | not less than 0.64 | not less than 0.57 |
| 9 | (Methanol:acetone::50:50) extract | not less than 0.39 | not less than 0.35 |
| 10 | (Methanol:chloroform::10:90) extract | not less than 1.74 | not less than 0.62 |
| 11 | (Methanol:chloroform::20:80) extract | not less than 2.28 | not less than 1.19 |

### Example 15

### Biological Effects

(a) Method for PBMC (Peripheral Blood Mononuclear Cells) Separation

Blood (20 ml) was collected from healthy volunteers in a heparanised vial with 10 ml of lymphoprep (Nycomed pharma, AS) and centrifuged at 1800 rpm for 30 minutes at 23° C., mononuclear cell containing buffy layer was transferred to another tube and washed three times with PBS (phosphate buffered saline) and centrifuged at 1500 rpm for 15 minutes. Pellet cells were resuspended in 2 ml of RPMI-1640 medium (Biochrom, AG) supplemented with 25 mM HEPES [N-(2-hydroxyethyl)piperazine-N′-(2-ethanesulfonic acid)], L-glutamine (2 mM), penicillin (100 U/ ml), streptomycin (100 μg/ml) and 10% inactivated FCS (fetal calf serum) and, finally viable cells were counted.

(b) In-vitro Mitogen Induced Lymphocyte Proliferation Assay (Mitogen Induced LPA)

PBM cells were adjusted to a concentration of 1×10⁶ cells/ml in RPMI buffer (Biochrom AG) and 2×10⁵ cells/well were seeded in a total volume of 200 μl to a 96 well

U bottom plate. Test samples (methanol extract; methanol and water (50:50) extract; water extract; acetone extract; chloroform fraction of methanolic extract; Boeravinone B and Boeravinone E) and control (buffered cells only) were set in triplicate in culture plate with and without phytohemagglutin or canavalin A as mitogen and incubated at 37° C. for 5 days in a CO₂ incubator containing 5% CO₂ and 90% humidity. Cultures were incorporated with 0.5 μCi[3H], 18 hours before the completion of incubation and cells were harvested on glass fiber filter using multi-well harvester and thymidine uptake was determined by measuring radioactivity on liquid scintillation counter and the mean count per minute (CPM) of triplicate was calculated.

DRC (dose response curve) using 0.1, 1, 10, 20, 50 and 100 μg/ml concentrations for methanol; methanol and water (50:50) and water extracts were obtained (FIG. 2). The three extracts, i.e. methanol; methanol and water (50:50) and water extracts displayed 84%, 88% and 55% of maximal inhibition against mitogen induced lymphocyte proliferation, respectively at the concentration of 100 μg/ml. The IC50 against this assay was between 10-20 μg/ml for the three extracts. Further, chloroform fraction of methanol extract resulted in 85% inhibition of mitogen induced lymphocyte proliferation at the maximal concentration of 100 μg/ml. The IC50 of chloroform fraction of methanolic extract against this assay was found to be 10-20 μg/ml. Boeravinone B and Boeravinone E isolated from chloroform fraction of methanolic extract exhibited a significant inhibition of 66 and 59% at 20 μg/ml concentration. An acetone extract (100 μg/ml), resulted in 80% inhibition of mitogen induced lymphocyte proliferation with an IC50 of 10 μg/ml.

(c) In-vitro Effect on Gene Expression of Inflammatory Mediators TNF-α, IL-1β and iNOS

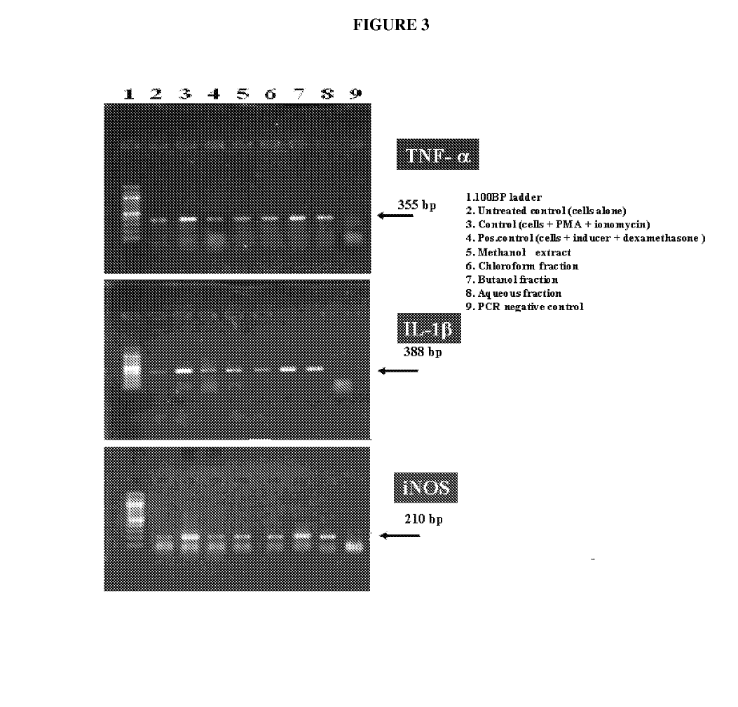

Methanol extract, chloroform fraction, butanol fraction and aqueous fraction of methanol extract were incubated with RAW 264.7 macrophage cell line for 12 hrs and expression of TNF-α, IL-1β and iNOS was seen using semi-quantitative RT-PCR (Reverse transcriptase Polymerase chain reaction) method. Incubation with methanol extract and its chloroform fraction resulted in down regulation of all the 3 inflammatory mediators (FIG. 3). Boeravinone B and Boeravinone E also down regulated all the 3 inflammatory mediators.

(d) In-vitro Nitric Oxide Release Assay

RAW 264.7 cells (Mouse leukaemic monocyte macrophage cell line) at a concentration of 25×10⁴ cells/ml in DMEM (Dulbecco's Modified Eagle's Medium; Biochrom AG), supplemented with 10% FCS (Fetal Calf Serum) were adjusted to a 96 well plate. Cells were incubated at 37° C. in a CO₂ incubator containing 5% CO₂ and 95% humidity for 48 hours. 100 μl of culture media from each well was replenished with same amount of fresh media. Test samples (methanol extract and chloroform fraction of methanol extract) and control (cultured cells without test sample) were set in triplicate in culture plate with and without LPS (lipopolysacchride) (0.5 μg/ml) and incubated at 37° C. for further for 24 hours. 100 μl supernatant from these culture wells were transferred to another plate and mixed with an equal volume of greiss reagent (1% sulfanilamide and 0.1% napthyl ethylene diamine dihydrochloride in 2.5% orthophosphoric acid) at room temperature for 10 minutes. Absorbance was determined at 57 nm in a microtiter plate reader. Nitric oxide concentration was estimated from a standard curve plotted using known quantity of sodium nitrite. Cultures were incorporated with 0.5 μCi[3H], 18 hours before the completion of incubation and cells were harvested on glass fiber filter using multi-well harvester and thymidine uptake was determined by liquid scintillation counter and the mean count per minute (CPM) of triplicate was calculated. Results were expressed in μM concentration obtained from mean OD (optical density) of triplicate of each sample.

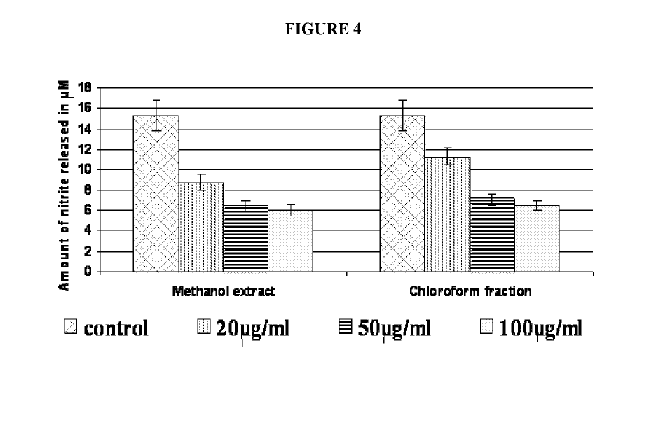

Dose response curves for methanol extract and chloroform fraction of methanol extract were obtained for the inhibition of NO release from RAW 264.7 cells (FIG. 4). Both methanol extract and its chloroform fraction moderately reduced the production of NO from RAW 264.7 cells. The IC50 for both methanol extract and chloroform fraction was less than 20 μg/ml.

(e) In-vitro Leukotriene B₄ (LTB₄) Release Assay

Neutrophils were isolated from freshly drawn human blood after dextran sedimentation and ficoll separation [Hatzelmann and Ullrich, Eur. J. Biochem., 169 (1987), 175-184]. 180 μl of neutrophil suspension (0.2×10⁶ cells/ml) was taken and was added with 19 μl of Hank's buffer salt solution along with 1 μl of the test samples (i.e. methanol extract; chloroform fraction of methanol extract; Boeravinone B, Boeravinone E, acetone extract and chloroform:methanol (90:10) extract) (200 times concentrated) in a 24 well plate and incubated at 37° C. for about 1 hour. Ca⁺⁺/Mg⁺⁺ (0.25 mM) was added, 3 minutes before the end of incubation with these test samples. Then, 0.3 μg/ml of A23187 (Calcimycin) was added and incubated for further 10 minutes. The reaction was stopped by adding 80 μl of cold methanol and mixture was spun at 3500 rpm for 10 minutes to remove cell debris. The samples were analyzed for LTB₄ assay [Hatzelmann and Schudt, J. Pharmacol. Exp. Ther., 297 (1): (2001), 267-279] using LTB₄ ELISA kits (Assay Design Inc., USA). The amount of LTB₄ was quantified and percent inhibition in LTB₄ release was calculated with respect to control (without test sample) to compute IC₅₀ values.

Methanol extract and chloroform fraction of methanol extract significantly inhibited the LTB₄ release from the PBM cells. IC50 of methanol and chloroform fraction of methanol extract for LTB₄ release from PBM cells was 93.1 and 100 μg/ml, respectively. Boeravinone E also significantly inhibited LTB₄ release with an IC50 of 21.8 and IC50 for Boeravinone B was more than 100 μg/ml. In-vitro, acetone extract and chloroform:methanol (90:10) extract markedly inhibited the LTB₄ release from PBM cells and the IC50 was 3.23 μg/ml and 48.1 μg/ml, respectively.

(f) Ex vivo LTB₄ Release Assay

Wistar rats were treated orally with various doses of acetone extract (10, 30 and 100 mg/kg) or vehicle (polyethylene glycol+water 20:80) and after 1 hour of administration, blood was withdrawn and freshly drawn blood of each group was challenged with A23187 (Calcimycin) separately and released LTB₄ was estimated using the ELISA kit, (assay designs, USA).

Oral administration of acetone extract at the doses of 10, 30 and 100 mg/kg to wistar rats resulted in 17, 46 and 42% inhibition of LTB₄ release, respectively.

(g) In-vitro TNF-α and IL-1β Release Inhibition Assay from Peripheral Blood Mononuclear Cells (PBMCs)

PBM cells were adjusted to 1×10⁶ cells/ml number in RPMI 1640 medium, (Biochrom AG) and 100 μl volume of this cell suspension per well was plated in a 96 well plate. 10 mM stock solutions of methanol extract; chloroform fraction of methanol extract; acetone extract; chloroform:methanol (90:10) extract; Boeravinone B and Boeravinone E were prepared by dissolving in dimethyl sulfoxide (DMSO) and the desired 10× dilutions were made with RPMI 1640. Twenty μl of DMSO control and each concentration of these prepared test samples were added to corresponding wells in a 96 well plate. The plate was incubated for 30 minutes at room temperature on rotatory shaker at 200 rpm and 50 μl of lipopolysacchride (LPS) (4 μg/ml) was added to each well, except negative LPS control wells. Plate was further incubated for 15 minutes at room temperature on rotatory shaker at 200 rpm and 30 μl of RPMI 1640 with 10% FCS (fetal calf serum) was added to all wells to make up the volume to 200 μl. Plate was overnight incubated at 37° C. in a CO₂ incubator and at the end of incubation period, plate was centrifuged at 3000 rpm for 10 minutes at 4° C., supernatant was separated and TNF-α and IL-1β estimation was done using a commercially available ELISA kit following the instructions given in the kit insert. A dose response curve was generated with different concentrations of test sample and the IC₅₀ was calculated using Graph Pad Prism. All test samples were assayed in duplicate.

In-vitro, methanol extract; chloroform fraction of methanol extract; acetone extract; chloroform:methanol (90:10) extract; Boeravinone B and Boeravinone E significantly inhibited the TNF-α release from these PBM cells and IC50 for these were 19.8; 16.1; 6.0; 10.0; 6.8 and 7.0 μg/ml, respectively. Boeravinone B and Boeravinone E also significantly inhibited IL-1β release from the PBM cells. The IC50 against IL-1β release was 3.6 and 7.0 μg/ml for Boeravinone B and Boeravinone E, respectively.

(h) Cell Viability/Toxicity Tests

Viability of cells was analyzed using (3-4,5-dimethylthiazol-2-yl)-2,5-diphenyl-tetrazolium bromide (MTT) assay (Mosmann T, J Immunol. Meth., 65 (1983), 55-63) with supernatant cells by adding 0.1 mL of MTT (0.25 mg/mL) with 0.1 mL of supernatant cells. The cells were incubated at 37° C. for about 2-4 hours, and then the optical density was measured at 490-650 nm.

Test samples (methanol extract, chloroform fraction of methanol extract, acetone extract, Boeravinone B and Boeravinone E) showed cytotoxcity less than 15% at 100 μg/ml concentration. Boeravinone B and Boeravinone E displayed 50% viability of tested cells at 64.5 and 81.5 μg/ml, respectively.

(i) Phenyl-p-Benzoquinone Induced Writhing Test in Mice for Analgesic Efficacy

Swiss albino mice were given orally the chloroform fraction of methanol extract (100, 300 and 1000 mg/kg) or vehicle (polyethylene glycol+water 20:80) or Indomethacin (5 mg/kg) and 1 hour after the treatment, the mice were challenged with 2 mg/kg of 0.2% solution of phenyl-p-benzoquinone (PQ). The writhing response (stretching, twisting hind leg inward and abdominal contraction) were counted from 5 to 20 minutes after PQ injection.

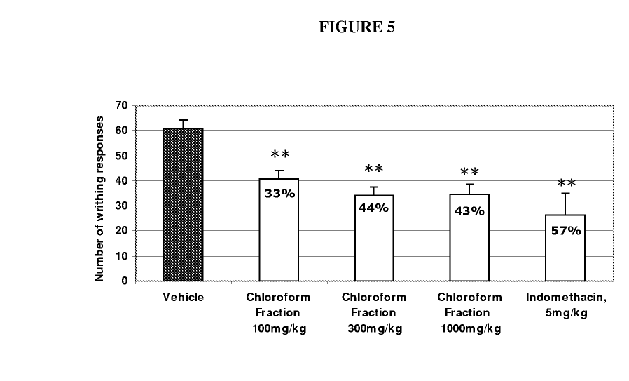

Single oral administration of chloroform fraction of methanol extract resulted into inhibition of PQ induced writhing in mice and a significant (p<0.01) inhibition was obtained with different test doses (FIG. 5).

(j) Analgesic Efficacy Against Carrageenan Induced Hyperalgesia in Rats

Chloroform fraction of methanol extract (30, 150, 300 and 1000 mg/kg) or vehicle (polyethylene glycol+water 20:80) or Indomethacin (5 mg/kg) were given orally to normal wistar rats and after 1 hour of administration, the weight bearing threshold of one hind leg was measured Immediately after measuring the basal weight bearing threshold of hind leg; 0.1 ml of 1% carrageenan was administered in the same leg and after two and three hours of carrageenan injection, again the weight bearing threshold of leg was measured and the change in weight bearing threshold was calculated.

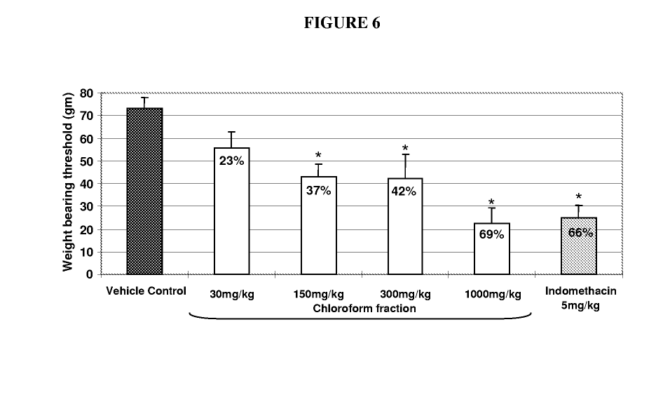

Single oral administration of chloroform fraction of methanol extract resulted into a dose dependent inhibition of carrageenan induced hyperalgesia in rats (FIGS. 6) and 1 g/kg dose of chloroform fraction of methanol extract produced a highest inhibition of weight bearing threshold of 69% after 2 hours of carrageenan challenge, whereas treatment with standard drug Indomethacin (5 mg/kg) produced a significant inhibition of 66% after 2 hours of administration.

(k) Analgesic Efficacy Against Formalin Test in Mice

Mice were treated with vehicle (polyethylene glycol+water 20:80) or chloroform fraction (100 and 300 mg/kg) orally or standard drug Pentazocin (10 mg/kg) intraperitoneally (i.p). Thirty minutes later, formalin solution (1% w/v, 25 μL/mice) was injected subplantarly in mice and immediately after injection, duration of mice licking or flicking of the injected paw was recorded for a period of 30 minutes in 5 minute slots. The first five minutes duration constitutes Phase-I pain which is centrally mediated while Phase-II pain which lasts from 10-30 minutes is peripherally mediated pain. Values of each group were expressed as mean±SEM (standard error of mean) and comparison was made with control group using one-way ANOVA followed by Dunnett's multiple test and a p≦0.05 was considered statistically significant.

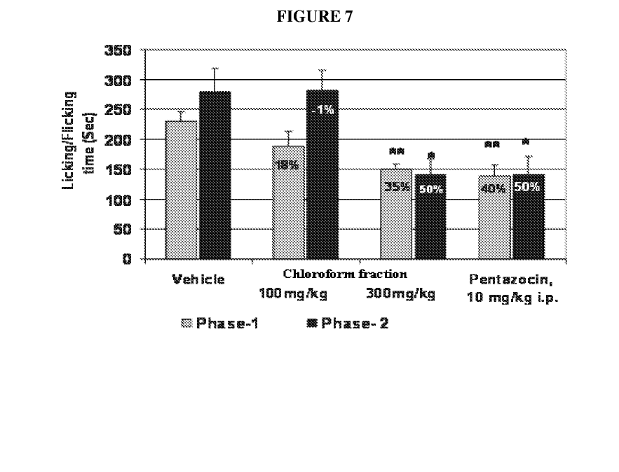

In vehicle treated group the duration of licking/flicking response in Phase-I and Phase-II was 230.7±15.1 and 279.9±37.7 sec., respectively. Oral treatment with chloroform fraction showed significant reduction in duration of both the phases at a dose of 300 mg/kg as compared to vehicle control group. Pentazocin at 10 mg/kg showed 40 and 50 percent protection in Phase-I and Phase-II pain, respectively, which was statistically significant (FIG. 7).

(l) Antihyperalgesic Activity Against Complete Freund's Adjuvant (CFA) Induced Hyperalgesia in Rats

Basal weight bearing threshold of the rats was determined using Randall Selitto analgesiometer (Ugo Basile, Italy) followed by challenge with Complete Freund's adjuvant (a suspension of desiccated Mycobacterium butyricum in a mixture of paraffin oil and an emulsifying agent, mannide monooleate; 50 μL of 500 μg/ml suspension/animal), injected subplantarly. Twenty four hours later weight-bearing threshold of the animals was recorded as pre-dose values at time 0 hours. Rats were either treated with vehicle (polyethylene glycol+water 20:80) or chloroform fraction of methanol extract (100 and 300 mg/kg) or Indomethacin (5 mg/kg) orally. Two hours post treatment, weight-bearing threshold of the animals was recorded again. Change in weight bearing threshold 2 hours post treatment in each treatment group was compared with the vehicle control group using one-way ANOVA followed by Dunnett's multiple comparison test and a p≦0.05 was considered statistically significant. One group was treated with Incomplete Freund's adjuvant (IFA) and served as negative control group.

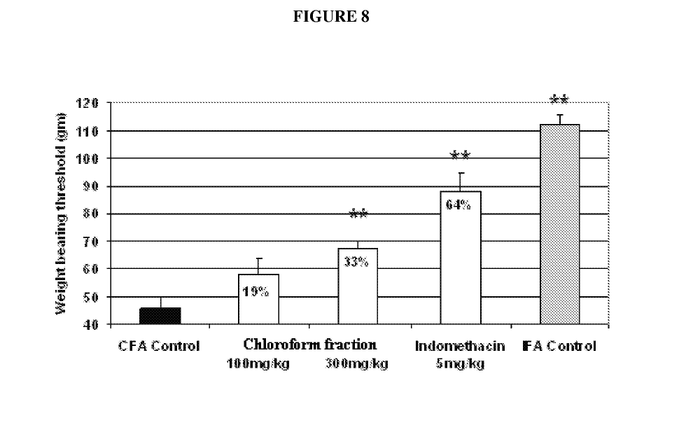

As shown in FIG. 8, weight bearing threshold of CFA challenged animals was decreased from the baseline as compared to IFA (Incomplete Freund's adjuvant) control animals. After 2 hours of treatment, weight bearing threshold of CFA challenged animals was further decreased to 45.60±4.28 from 60±3.9 gms, however a marginal increase in weight bearing threshold was observed after two hours of treatment in IFA control group. Treatment with chloroform fraction showed significant improvement in weight bearing threshold at 300 mg/kg dose as compared to vehicle control group (FIG. 8). Indomethacin produced a 64% reversal of the hyperalgesia induced by CFA. This effect assumed significance as in this therapeutic model; chloroform fraction was able to reverse a pre-existing pain.

(m) Anti-inflammatory Efficacy Against Carrageenan Induced Paw Edema Model

Rats were treated orally with vehicle (polyethylene glycol+water 20:80) or chloroform fraction (10, 100 and 300 mg/kg) and Indomethacin (5 mg/kg). One hour later, animals were challenged with carrageenan (1% w/v, 100 μL/rat) subplantarly. The paw edema was recorded 3 hours post carrageenan challenge. Data from each group was expressed as mean±SEM. Change in paw edema in treatment group were compared from vehicle control group using one-way ANOVA followed by Dunnett's multiple comparison test. A p≦0.05 was considered statistically significant.

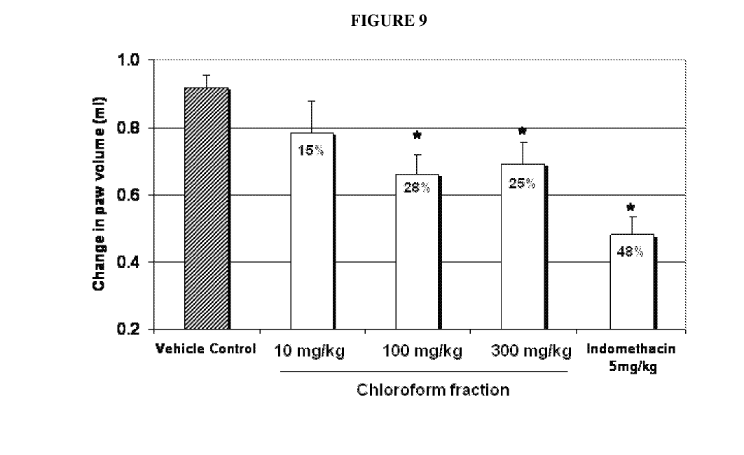

Carrageenan injection produced a 0.92±0.09 mL increase in the paw volume in 3 hrs. Treatment with chloroform fraction significantly inhibited carrageenan induced paw edema, 28 and 25%, at 100 and 300 mg/kg respectively (FIG. 9). Treatment with Indomethacin (5 mg/kg) produced a significant inhibition of 48%.

(n) Anti-Inflammatory Efficacy Against Endotoxemia in Female Balb/C Mice

Mice were treated orally with vehicle (polyethylene glycol+water 20:80) or chloroform fraction (10, 30, 100 and 300 mg/kg) or Vx745 (10 mg/kg). One hour later, animals were challenged with LPS (lipopolysacchride). Ninety minutes later, blood was collected and TNF-α was estimated in the plasma using ELISA kit and values for each treatment group was expressed as mean±SEM. Comparison was made with control group using one-way ANOVA followed by Dunnett's multiple test and a p≦0.05 was considered statistically significant.

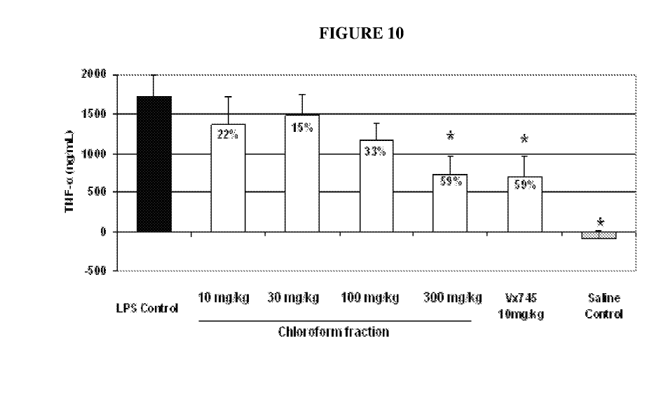

LPS challenge in mice produced a significantly more TNF-α release as compared to saline challenged mice. Chloroform fraction produced a dose related inhibition of LPS induced TNF-a release with a significant inhibition of 59% seen at dose of 300 mg/kg; and method control substance Vx745 (10 mg/kg) showed a significant inhibition (59%) of LPS response (FIG. 10).

(o) Anti-Inflammatory Efficacy in Air Pouch Model in Rats

A pneumoderma was made in the middle of the dorsal skin of rat by injecting 20 ml of sterile air on day zero followed by injection of additional 10 mL on day 3 in the resulting oval air pouch. On day 5, rats were treated orally with vehicle (polyethylene glycol+water 20:80) or chloroform fraction of methanol extract (100, 300 and 1000 mg/kg), one hour later, carrageenan was injected (0.5% w/v, 2 ml/rat) into the pouch. Four hours after carrageenan challenge, animals were euthanized and the pouch was lavaged with ice-cold Hank's balanced salt solution. TNF-α was estimated in the supernatant of lavage fluid and the values were expressed as mean±SEM for each group. Comparison was made between treatment group and vehicle control group using one-way ANOVA followed by Dunnett's multiple test. A p<0.05 was considered statistically significant.

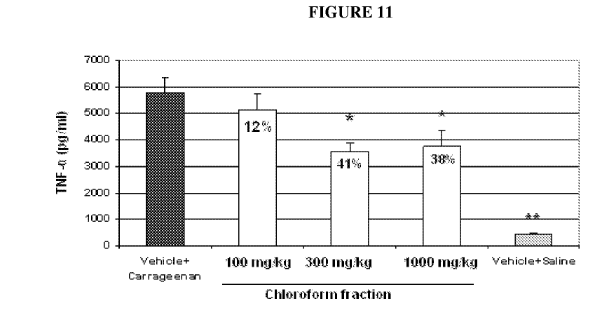

In vehicle treated group challenged with carrageenan, challenge caused a significant increase of more than 14 fold in TNF-α release as compared to the saline challenge. Treatment with chloroform fraction 100 mg/kg produced marginal decrease in TNF-α release; however treatment with higher doses of 300 and 1000 mg/kg showed a 41% and 38% inhibition, respectively, which was statistically significant as compared to carrageenan control group (FIG. 11).

(p) Antiarthritic Efficacy Against Complete Freund's Adjuvant (CFA) Induced Arthritis Model in Rats

Wistar rats were injected with 50 μL of 1 mg/ml solution of Complete Freund's adjuvant (CFA) subplantarly. Day 10 onward the contra-lateral paw was observed for any change in paw swelling. The animals showing 0.30-0.40 ml increase in paw volume of the contralateral paw from the basal were included in the study.

The animals were treated with chloroform fraction of methanol extract at dose of 300, 600 and 1200 mg/kg/day, p.o. (per oral) and Indomethacin at 0.2 mg/kg/day was dosed as standard control. The test sample or vehicle (polyethylene glycol+water 20:80) was administered in two divided doses for 10 days. The paw volume of the animals was recorded on the alternate days. A plot of change in paw volume from day 0 was made and area under the curve (AUC) was calculated using GraphPad Prism software (GraphPad Software Inc, USA, Version 4) for each animal and was averaged in each treatment group.

Effect on the progression of the arthritis, measured as AUC, observed in different treatment groups was compared with vehicle control group using one-way ANOVA followed by Dunnett's multiple comparison tests and a p≦0.05 was considered statistically significant.

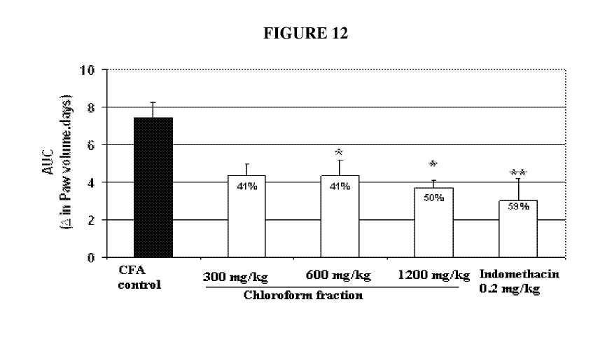

Subplantar injection of Complete Freund's adjuvant produced an acute inflammation in the injected paw. This was followed by induction of inflammation in the un-injected contra-lateral paw from days 11-14, which continued to increase further and a peak increase of 1±0.1 mL was observed on day 6 post inductions. The AUC in vehicle treated group challenged with CFA was 7.41±0.84. Treatment with chloroform fraction showed a significant improvement in paw edema, 6 days onwards at the dose of 600 and 1200 mg/kg/day, when compared to vehicle control. This was reflected in a significant inhibition of AUC at these doses as seen in the FIG. 12. However, Indomethacin at 0.2 mg/kg showed reversal of the paw edema toward basal level.

(q) Histopathological Analysis of CFA Induced Arthritis in Ankle Joint of Rats

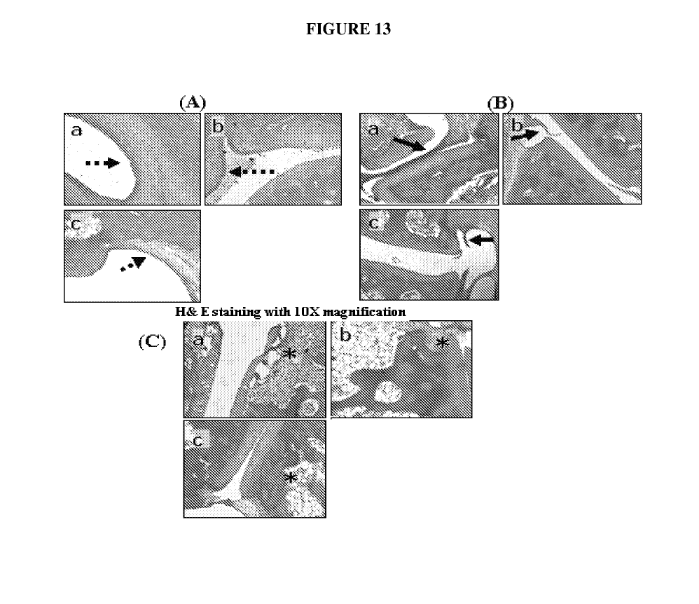

At the end of the study (p), animals were euthanized and ankle joints of contra-lateral hind limb were collected for histopathological evaluation Ankle joints were evaluated for the effect on synovial proliferation, inflammation of soft tissues, pannus formation and erosion of cartilage as well as bone (FIG. 13). The changes observed were scored as 0=no change, 1 mild, 2=moderate, 3=marked and 4=severe. The scores for each parameter mentioned above were summed to obtain total histopathological score for each animal. Histopathological scores for each treatment group were compared with vehicle control group using one-way ANOVA followed by Dunnett's multiple comparison test and a p≦0.05 was considered statistically significant.

In vehicle treated group, a histopathological score of 6.9±0.8 (Table 1) was obtained which showed mild to moderate inflammation of soft tissue, pannus formation and moderate synovial proliferation as well as moderate to marked erosion of bone and cartilage tissue of the ankle joint.

Treatment with chloroform fraction at 600 and 1200 mg/kg/day also showed a significant reversal of the CFA induced arthritic changes with statistically significant inhibition of 46.3 and 47.3% respectively (Table 1). These arthritic changes were significantly reversed by Indomethacin treatment with a total score of 2.56±0.6 with an inhibition of 62.8% as shown in Table 1.

TABLE 1
Effect of treatment on histopathological score of
ankle joints of adjuvant induced arthritic rats

| | Histopathological | % |
|---|---|---|
| Treatment | scores | inhibition |
| CFA Control | 6.9 ± 0.8 | — |
| Chloroform fraction 300 mg/kg | 3.7 ± 1 | 46.0 |
| Chloroform fraction 600 mg/kg | 3.7 ± 0.8* | 46.3 |
| Chloroform fraction 1200 mg/kg | 3.6 ± 0.3* | 47.3 |
| Indomethacin 0.2 mg/kg | 2.56 ± 0.6** | 62.8 |

wherein
*signifies p < 0.05;
**signifies p < 0.01

We claim:

1. A standardized extract of Boerhaavia diffusa.

2. The standardized extract of claim 1, comprising of bioactive markers Boeravinone B and Boeravinone E.

3. The standardized extract of claim 2 wherein the percentage content of Boeravinone B is 0.1%-4.0% and that of Boeravinone E is 0.05%-3.0%.

4. A pharmaceutical composition comprising Boeravinone B, Boeravinone E, or the standardized extract of claims 1 to 3, along with one or more of pharmaceutically acceptable carriers, excipients or diluents.

5. A process for the isolation of Boeravinone B and Boeravinone E from Boerhaavia diffusa, the process comprising
a) extracting the plant mass of Boerhaavia diffusa with a solvent,
b) concentrating the extract,
c) adding water to extract,
d) partitioning the extract with a solvent, and
e) isolating Boeravinone B and Boeravinone E.

6. The process of claim 5, wherein the extraction solvent is selected from the group consisting of alcohol, ketone, ester, halogenated hydrocarbon, nitrile and mixture(s) thereof.

7. The process of claim 5, wherein the partitioning solvent is selected from the group consisting of halogenated hydrocarbon, ester, alcohol, ether and mixture(s) thereof.

8. A process for the preparation of extracts of Boerhaavia diffusa enriched with bioactive markers, the process comprising
a) extracting the plant mass of Boerhaavia diffusa with a solvent selected from the group consisting of alcohol, ketone, ester, halogenated hydrocarbon, water and mixture(s) thereof, and
b) drying the extract.

9. A process for the preparation of extracts of Boerhaavia diffusa enriched with bioactive markers, the process comprising extracting the plant mass of Boerhaavia diffusa with a solvent,
a) adding water to extract,
b) partitioning the extract with a solvent,
c) drying the extract.

10. The process of claim 9, wherein the extraction solvent is selected from the group consisting of alcohol, ketone, ester and mixture(s) thereof.

11. The process of claim 9, wherein the solvent in step b) is selected from the group consisting of halogenated hydrocarbon, ester, alcohol, ether and mixture(s) thereof.

12. A method for the standardization of extracts of Boerhaavia diffusa, the method comprising detecting and quantifying bioactive markers.

13. A standardized extract of Boerhaavia diffusa prepared by a method comprising
a) extracting the plant mass of Boerhaavia diffusa with a solvent,
b) drying the extract,
c) standardizing the extract by using bioactive markers.

14. The method of claim 13, wherein the solvent is selected from the group consisting of alcohol, ketone, ester, halogenated hydrocarbon, water and mixture(s) thereof.

15. A standardized extract of Boerhaavia diffusa prepared by a method comprising
a) extracting the plant mass of Boerhaavia diffusa with a solvent,
b) adding water to extract,
c) partitioning the extract with a solvent,
d) drying the extract,
e) standardizing the extract by using bioactive markers.

16. The method of claim 15, wherein the solvent in step a) is selected from the group consisting of alcohol, ketone, ester and mixture(s) thereof.

17. The method of claim 15, wherein the solvent in step c) is selected from the group consisting of halogenated hydrocarbon, ester, alcohol, ether and mixture(s) thereof.

18. The method of claim 12, 13 or 15, wherein bioactive marker is Boeravinone B, Boeravinone E, or mixture(s) thereof.

19. A method of treating inflammatory diseases in a mammal comprising administering a therapeutically effective amount of Boeravinone B, Boeravinone E, or a standardized extract of Boerhaavia diffusa.

20. A method of treating pain in a mammal comprising administering a therapeutically effective amount of Boeravinone B, Boeravinone E, or a standardized extract of Boerhaavia diffusa.

21. A method of treating rheumatoid arthritis, osteoarthritis, acute myoskeletal disorders, spondylosis, ankylosing spondylitis, bursitis, tendonitis, inflammatory lung disease, inflammatory bowel disease, atherosclerosis, systemic lupus erythematosus, multiple sclerosis, pelvic inflammatory disease or psoriasis in a mammal comprising administering a therapeutically effective amount of Boeravinone B, Boeravinone E, or a standardized extract of Boerhaavia diffusa.

22. A method of treating dental pain, muscular pain, neck pain, ear pain, joints pain, headache, abdominal pain, renal pain, pelvic pain, prolapsed intervertebral disc pain or neuropathic pain in a mammal comprising administering a therapeutically effective amount of Boeravinone B, Boeravinone E, or a standardized extract of Boerhaavia diffusa.
